(19) United States
(12) Patent Application Publication
(10) Pub. No.: US 2023/0414468 A1
(43) Pub. Date: Dec. 28, 2023

(54) WASH COMPOSITION

(71) Applicant: Conopco, Inc., d/b/a UNILEVER, Englewood Cliffs, NJ (US)

(72) Inventors: Bivash Ranjan DASGUPTA, Cheshire, CT (US); Vickie J FOY, Newtown, CT (US); Teanoosh MOADDEL, Watertown, CT (US)

(73) Assignee: Conopco, Inc., d/b/a UNILEVER, Englewood Cliffs, NJ (US)

(21) Appl. No.: 18/036,268

(22) PCT Filed: Nov. 5, 2021
(86) PCT No.: PCT/EP2021/080746

§ 371 Date: May 10, 2023

(51) Int. Cl. A61K 8/44; A61Q 19/00; A61K 8/46; A61K 8/73; A61K 8/60; A61Q 19/10

(52) U.S. Cl. CPC A61K 8/442; A61Q 19/005; A61K 8/466; A61K 8/731; A61K 8/602; A61Q 19/10; A61K 2800/75; A61K 2800/596; A61K 2800/5426

(57) ABSTRACT

The invention is directed to a wash composition that does not irritate the eyes. The composition comprises a surfactant system having anionic surfactant, and amphoteric or zwitterionic surfactant or both, and nonionic surfactant or cationic polymer or a mixture thereof. The composition is water continuous, has a pH from 4.8 to 7.5, and surprisingly, does not irritate the eyes while simultaneously delivering consumer desired lathering characteristics.

### FIELD OF INVENTION

The present invention is directed to a wash composition that does not irritate the eyes. More particularly, the composition is a wash composition comprising a surfactant system having anionic surfactant, and amphoteric or zwitterionic surfactant or both, and nonionic surfactant or cationic polymer or a mixture thereof. The composition is water continuous, has polyol, a pH from 4.8 to 7.5, and surprisingly, does not irritate the eyes while simultaneously delivering consumer desired skin benefits and lathering characteristics. The composition of the present invention is gentle enough for consumers with compromised and/or weak skin barriers, including babies.

### BACKGROUND OF THE INVENTION

Normally, wash compositions are desired for use by consumers not only for cleansing and sanitizing but also to deposit skin benefit agents such as moisturizing agents, antibacterial agents and fragrances on to the skin. Given the current state of the world, one in a pandemic, there is a strong desire to wash more often, especially the hands and face. Consumers, in view of this, require products that are gentle to use and that deliver an adequate amount of lather upon washing. Consumers also demand that the wash compositions they use are non-irritating to the eyes, especially when it is increasingly important to wash the face. This is particularly true when consumers are tending to young ones, and especially, babies.

It is of increasing interest to deliver wash compositions that cleanse and disinfect well, provide skin benefit agents, are gentle to use and that provide good lathering without causing eye irritation. The present invention, therefore, is directed to a composition that comprises a surfactant system having anionic surfactant, and amphoteric or zwitterionic surfactant or both, and nonionic surfactant or cationic polymer or a mixture thereof. The composition is water continuous, has a pH from 4.8 to 7.5, and surprisingly, is not irritating to the eyes while simultaneously delivering consumer desired lathering characteristics.

Additional Information Efforts have been disclosed for making wash compositions. In U.S. Pat. No. 4,221,733, wash compositions with betaines and that display reduction in eye irritation are described.

Other efforts have been disclosed for making wash compositions. In U.S. Pat. No. 6,169,060, cleanser compositions with anionic, nonionic and amphoteric surfactants are described.

Still other efforts have been disclosed for making wash compositions. In U.S. Pat. No. 6,372,934, water soluble compounds that do not irritate the eyes and skin and are suitable for use in personal care compositions are described.

None of the additional information describes a wash composition that is gentle on the eyes as defined by the claimed invention.

### SUMMARY OF THE INVENTION

In a first aspect, the present invention is directed to a wash composition comprising:
a) a surfactant component comprising:
   i) amphoteric and/or zwitterionic surfactant;
   ii) nonionic surfactant, cationic polymer or a mixture thereof; and
   iii) anionic surfactant, the anionic surfactant comprising from 25 to 100% by weight taurate based on total weight of anionic surfactant;
b) from greater than 5 to 55% by weight polyol; and
c) water
   wherein the amphoteric and/or zwitterionic surfactant, and nonionic surfactant or cationic polymer or mixture thereof (“component i and ii”, respectively) to anionic surfactant (“component 30 iii”) are at a weight ratio from 1:4 to 2:1 and the amphoteric and/or zwitterionic surfactant (“component i”) to nonionic surfactant, or cationic polymer or mixture thereof (“component ii”) are at a weight ratio from 1:12 to 12:1, with the proviso that when cationic polymer is used alone or with nonionic surfactant, the total weight of cationic polymer used to total weight of surfactant in the wash composition is from 1.3 to no more than 15% by weight of total weight of surfactant and the wash composition comprises 20% or less by weight total of surfactant and cationic polymer.

In a second aspect, the present invention is directed to a method for washing with the wash composition of the first aspect of the present invention.

In a third aspect, the invention is directed to the use of the composition of the first aspect of the present invention to wash the face and/or hair without irritating the eyes.

All other aspects of the present invention will more readily become apparent from the description and examples which follow.

Skin, as used herein, is meant to include skin on the arms (including underarms), face, feet, neck, chest, hands, legs, buttocks and scalp (including hair). Irritating to the eye, as used herein, means a stinging or burning sensation as determined by panelist self-assessment of the eyes subjected to wash composition. It also includes visible redness assessment of a panelist's conjunctiva of the eye (by a trained professional) and/or developing irritation or lacrimation in the eye within 30 seconds to one (1) hour after contacting the eye with an aqueous composition comprising a 5% solution of wash composition. Such a wash composition is suitable to be a shampoo, conditioner, 3-in-1 wash composition (shampoo, conditioner & body wash), 2-in-1 wash composition (shampoo & body wash), make-up wash, facial wash, hand wash or personal care liquid body wash. Preferably, the wash composition of the present invention is a body or face wash that is gentle enough to apply to skin, including baby skin, ready for topical application and to be wiped or washed off, and preferably, washed off, with water. The wash composition may, optionally, comprise medicinal or therapeutic agents, but preferably, the composition is a wash which is a cosmetic and non-therapeutic wash for removing soil and/or bacteria often associated with odor. In an embodiment of the invention, the wash composition is a personal wash composition especially suited for use on babies. The wash composition of the present invention may optionally comprise skin benefit ingredients added thereto such as vitamins (like vitamins A, D, E and K, and especially, vitamin C and/or niacinamide) and/or derivatives thereof, resorcinols, retinoic acid precursors, colorants, moisturizers, sunscreens, mixtures thereof or the like. The skin benefit ingredients (or agents) may be water or oil soluble. If used, oil soluble skin benefit agents typically make up to 1.5% by weight of the wash composition whereby water-soluble skin benefit agents, when used, typically make up to 10% by weight of the wash composition. The wash composition typically has a pH from 4.8 to 7.5, and preferably, 5 to 7.5, and most preferably, 5.5 to 7.5. In an embodiment of the invention, the pH of the wash composition can be 6.0 to 7.2, including all ranges subsumed therein. Viscosity, unless noted otherwise, is taken with a Discovery HR-2 Rheometer using sand blasted plates having a 1000 micron gap and a first shear rate S_{A} of 0.4 s⁻¹ for a first viscosity V_{A} and a second shear rate S_{B} of 10 s⁻¹ for a second viscosity V_{B}, both at 25° C. and 20 second intervals. Viscosity is reported in centipoise (cps) (1000 centipoise (cps)=1 Pascal second). Stable, as used herein, means no discoloration or phase separation of the wash composition after being stored for at least one (1) month at 45° C., and preferably, from 2 to 4 months at 45° C. The term comprising is meant to encompass the terms consisting essentially of and consisting of. For the avoidance of doubt, and for illustration, the composition of this invention comprising surfactant and water is meant to include a composition consisting essentially of the same and a composition consisting of the same. Except in the operating comparative examples, or where otherwise explicitly indicated, all numbers in this description indicating amounts or ratios of materials or conditions and/or physical properties of materials and/or use are to be understood as modified by the word “about”. All ranges defined herein are meant to include all ranges subsumed therein unless otherwise stated.

### DETAILED DESCRIPTION OF THE INVENTION

Amphoteric surfactants suitable for use in the invention (which depending on pH can be zwitterionic) include sodium acyl amphoacetates, sodium acyl amphopropionates, disodium acyl amphodiacetates and disodium acyl amphodipropionates where the acyl (i.e., alkanoyl group) can comprise a C₇-C₁₈ alkyl portion. Illustrative examples of the amphoteric surfactants suitable for use include sodium lauroamphoacetate, sodium cocoamphoacetate, potassium lauroamphoacetate, potassium cocoamphoacetate or mixtures thereof. Sodium lauroamphoacetate is the preferred amphoteric surfactant used. When used, amphoteric surfactant typically makes up from 0.5 to 7%, and preferably, from 1 to 6%, and most preferably, from 1 to 5% by weight of the wash composition, including all ranges subsumed therein.

As to the zwitterionic surfactants that may be employed in the wash composition of the present invention, such surfactants include at least one acid group. Such an acid group may be a carboxylic or a sulphonic acid group. They often include a quaternary nitrogen, and therefore, can be quaternary amino acids. They should generally include an alkyl or alkenyl group of 7 to 18 carbon atoms and generally comply with an overall structural formula:

R¹—[—C(O)—NH(CH₂)_{q}—]ᵣ—N⁺(R²)(R³)-A-B

where R¹ is alkyl or alkenyl of 7 to 18 carbon atoms; R² and R³ are each independently alkyl, hydroxyalkyl or carboxyalkyl of 1 to 3 carbon atoms; q is 2 to 4; r is 0 to 1; A is an alkylene of 1 to 3 carbon atoms optionally substituted with hydroxyl, and B is —CO₂— or —SO₃—.

Often desirable zwitterionic surfactants for use in the wash composition of this invention and within the above general formula include simple betaines of formula:

R¹—N(R²)(R³)—CH₂CO₂⁻

and amido betaines of formula:

R¹—CONH(CH₂)ₜ—N⁺(R²)(R³)—CH₂CO₂⁻

where t is 2 or 3.

In both formulae R¹, R² and R³ are as defined previously. R¹ may, in particular, be a mixture of C₁₂ and C₁₄ alkyl groups derived from coconut oil so that at least half, preferably at least three quarters of the groups R¹ have 10 to 14 carbon atoms. R² and R³ are preferably methyl.

A further possibility is that the zwitterionic surfactant is a sulphobetaine and/or a sultaine.

Illustrative yet nonlimiting examples of the zwitterionic surfactants suitable for use include betaines such as lauryl betaine, betaine citrate, cocodimethyl carboxymethyl betaine, cocoamidopropyl betaine, cocoalkyldimethyl betaine, and laurylamidopropyl betaine. Still other zwitterionic surfactants suitable for use include a cocoamidopropyl sultaine, like for example, cocamidopropyl hydroxysultaine. Preferred zwitterionic surfactants include lauryl betaine, betaine citrate, sodium hydroxymethylglycinate, (carboxymethyl) dimethyl-3-[(1-oxododecyl) amino] propylammonium hydroxide, cocoalkyldimethyl betaine, (carboxymethyl) dimethyloleylammonium hydroxide, cocoamidopropyl betaine, (carboxymethyl) dimethyloleylammonium hydroxide, cocoamidopropyl betaine, (carboxylatomethyl) dimethyl(octadecyl)ammonium, cocamidopropyl hydroxysultaine, or a mixture thereof. Such surfactants are made commercially available from suppliers like Stepan Company, Solvay, Evonik and the like and it is within the scope of the invention to employ mixtures of the aforementioned surfactants.

In an embodiment of the invention zwitterionic surfactants for use in the present invention include cocodimethyl carboxymethyl betaine, cocamidopropyl betaine and laurylamidopropyl betaine. Additional preferred zwitterionic surfactants suitable for use include cocamidopropyl sultaine and cocamidopropyl hydroxysultaine. In a most preferred embodiment, the zwitterionic surfactant used in this invention is cocamidopropyl betaine, cocamidopropyl hydroxysultaine or a mixture thereof. When used, zwitterionic surfactant typically makes up from 0.5 to 7%, and preferably, from 1 to 6%, and most preferably, from 1 to 5% by weight of the wash composition, including all ranges subsumed therein.

Nonionic surfactants may be used in the wash composition of the present invention. The nonionics which may be used include in particular the reaction products of compounds having a hydrophobic group and a reactive hydrogen atom, for example aliphatic alcohols, acids, amides or alkylphenols with alkylene oxides, especially ethylene oxide either alone or with propylene oxide. Specific nonionic surfactant compounds are alkyl (C₆-C₂₂) phenols, ethylene oxide condensates, the condensation products of aliphatic (C₈-C₁₈) primary or secondary linear or branched alcohols with ethylene oxide, and products made by condensation of ethylene oxide with the reaction products of propylene oxide and ethylenediamine. Other nonionic surfactants include long chain tertiary amine oxides, long chain tertiary phosphine oxides, dialkyl sulphoxides, and the like.

Preferred nonionics that may be used in the wash composition of the present invention are glucamides, polyglycerol esters or mixtures thereof. In an embodiment of the invention, the glucamides used in the present invention include lauroyl methyl glucamide, myristoyl methyl glucamide, cocoyl methyl glucamide, capryloyl/caproyl methyl glucamide, sunfloweroyl methyl glucamide mixtures thereof or the like. Preferred glucamides are sold commercially by Clariant under the GlucoTain® name.

In yet another embodiment of the invention, the polyglycerol ester used includes polyglyceryl-8 caprylate, polyglycerol-8 caprate, polyglyceryl-8 laurate, polyglyceryl-8 myristate, polyglyceryl-8 palmitate, polyglycerol-8 stearate, polyglycerol-8 oleate, polyglyceryl-8 cocoate, polyglyceryl-9 caprylate, polyglycerol-9 caprate, polyglyceryl-9 laurate, polyglyceryl-9 myristate, polyglyceryl-9 palmitate, polyglycerol-9 stearate, polyglycerol-9 oleate, polyglyceryl-9 cocoate, polyglycerol-10 caprylate, polyglycerol-10 caprate, polyglycerol-10 laurate, polyglycerol-10 myristate, polyglycerol-10 palmitate, polyglycerol-10 stearate, polyglycerol-10 oleate, polyglyceryl-10 cocoate, polyglycerol-11 caprylate, polyglycerol-11 caprate, polyglycerol-11 laurate, polyglycerol-11 myristate, polyglycerol-11 palmitate, polyglycerol-11 stearate, polyglycerol-11 oleate, polyglyceryl-11 cocoate, polyglycerol-12 caprylate, polyglycerol-12 caprate, polyglycerol-12 laurate, polyglycerol-12 myristate, polyglycerol-12 palmitate, polyglycerol-12 stearate, polyglycerol-12 oleate, polyglyceryl-12 cocoate or a mixture thereof.

When nonionic surfactant is used in the wash composition of the present invention, such surfactant typically makes up from 0.5 to 7%, and preferably, from 1 to 6%, and most preferably, from 1 to 5% by weight of the wash composition, including all ranges subsumed therein.

As to the cationic polymer suitable for optional use, the same includes polymers classified as cationic guar gum derivatives, synthetic cationic polymers, and cationic starch derivatives. Those classified as partially cationically substituted quaternary ammonium salts are often desired for use.

Suitable polymers of this type conform to the structure given below:

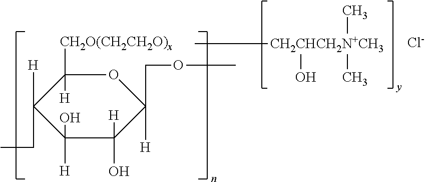

wherein, x is from 0 to 3, preferably from 0 to 1; and the ratio of y:n is in the range of from 0.01 to 0.5 (i.e. n:y=from 100 to 2). It is desirable in the present invention that cationic polymers with fully quaternized sugar units, i.e. y=n, are excluded.

The ratio of unquaternized to quaternized sugar units (n:y) is preferably in the range of from 3 to 30, and more preferably, from 4 to 25, most preferably from 5 to 20.

The weight average molecular weight of suitable polymers is in the range of from 100 to 3,000,000 kDa, preferably, from 500 to 1,000,000 kDa, more preferably, from 10,000 to 500,000 kDa, and mixtures of polymers may be used. In an embodiment of the invention, the weight average molecular weight of the polymer is from 150 to 100,000 kDa. In another embodiment, the weight average molecular weight of the polymer is from 200 to 3,000 kDa. In still another embodiment, the weight average molecular weight of the polymer is from 250 to 2,500 kDa.

Preferred cationic polymers for use are referred to as quaternary nitrogen-containing polysaccharides, more preferably, quaternary nitrogen-containing cellulose ethers, such as those described in U.S. Pat. Nos. 3,472,840; 3,962,418; 4,663,159, 5,407,919 and WO2005/000903 A1, the disclosures of which are incorporated herein by reference. Particularly preferred cationic polymers are quaternary nitrogen containing hydroxyethyl celluloses. Suitable examples of cationic polymers are salts of hydroxyethyl cellulose reacted with a trimethyl ammonium substituted epoxide, referred to in the industry by the Cosmetic, Toiletry, and Fragrance Association (CTFA) as Polyquaternium-10, the same being commercially available from Amerchol Corporation, a subsidiary of The Dow Chemical Company, as UCAREM Polymer JR-125, UCARE Polymer JR-400, UCARE Polymer KF, UCARE Polymer JR-30M, UCARE Polymer LR-400, UCARE Polymer LR-30M, and UCARE Polymer LK. Examples of other preferred cationic polymers are referred to by CTFA Polyquaternium-67. They are commercially available from Amerchol Corp. as the SoftCAT™ polymers like SoftCAT SL 5, SoftCAT SL 30, SoftCAT SL 60, SoftCAT SL 100, SoftCAT SK-L, SoftCAT SK-M, SoftCAT SK-M, SoftCAT SK-MH, SoftCAT SK-H, SoftCAT SX-400X, SoftCAT SX-400H, SoftCAT SX-1300X and SoftCAT SX-1300H. Other examples of preferred cationic polymers are those referred to in the industry by the CTFA as Polyquaternium-6 (i.e., PDADMAC, polydiallyldimethylammonium chloride), and Polyquaternium-7 with the CAS Registry Numbers 26062-79-3 and 026590-05-6, respectively, and those referred by the CTFA as Polyquaternium-44. Still other suitable polymers include Jaguar C13S, Jaguar C14S, and Jaguar C17 made commercially available from Solvay, and Merquat™ 100 from Lubrizol and the like. Even other types of cationic cellulose ethers include the polymeric quaternary ammonium salts of hydroxyethyl cellulose reacted with lauryl dimethyl ammonium-substituted epoxide referred to in the industry (CTFA) as Polyquaternium 24.

In an embodiment of the invention, cationic polymers used are those that yield in a 1% solution of cationic polymer and water a viscosity from 150 to 4000 cps, and preferably, from 200 to 3500 cps, and most preferably, from 300 to 2500 cps. In yet another embodiment, such a solution has a viscosity from 275 to 2100 cps, including all ranges subsumed therein.

As to the percent substitution of nitrogen by weight (i.e., cationic substitution) within the cationic polymer, typically the percent nitrogen is 0.1 to 4%, and preferably, from 0.3 to 3.5%, and most preferably, from 1 to 2.8% by weight, based on total weight of the cationic polymer.

Typically, when used, cationic polymer will make up from 1.3 to no more than 15% by weight of the total weight of surfactant used in the wash composition. Often cationic polymer makes up from 1.5 to 12%, and more often, from to 4 to 12% by weight of the total weight of surfactant used in the wash composition. In an embodiment of the invention, cationic polymer makes up from 5 to 11%, and preferably, from 6 to 10% by weight of the total weight of surfactant used in the wash composition.

Optionally, cationic surfactants may be included in the wash composition of the present invention. Those that may be used includes heterocyclic ammonium salts such as cetyl or stearyl pyridinium chloride, alkyl amidoethyl pyrrylinodium methyl sulfate, lapyrium chloride or mixtures thereof.

Tetra alkyl ammonium salts are another useful class of optional cationic surfactants for use. Examples include cetyl or stearyl trimethyl ammonium chloride or bromide; hydrogenated palm or tallow trimethylammonium halides; behenyl trimethyl ammonium halides or methyl sulfates; decyl isononyl dimethyl ammonium halides; ditallow (or distearyl) dimethyl ammonium halides, and behenyl dimethyl ammonium chloride.

Still other types of cationic surfactants that may be used are the various ethoxylated quaternary amines and ester quats. Examples include PEG-5 stearyl ammonium lactate (e.g., Genamin KSL manufactured by Clariant), PEG-2 coco ammonium chloride, PEG-15 hydrogenated tallow ammonium chloride, PEG-15 stearyl ammonium chloride, dipalmitoyl ethyl methyl ammonium chloride, dipalmitoyl hydroxyethyl methyl sulfate, and stearyl amidopropyl dimethylamine lactate.

Even other useful yet optional cationic surfactants include quaternized hydrolysates of silk, wheat, and keratin proteins, and it is within the scope of the wash composition to use mixtures of the aforementioned cationic surfactants.

If used, cationic surfactants will typically make up no more than 3% by weight of the total weight of surfactant in the wash composition. When present, cationic surfactants typically make up from 0.01 to 2.5%, and more typically, from 0.1 to 1.5% by weight of the total weight of surfactant used in the wash composition, including all ranges subsumed therein.

In an embodiment of the invention, cationic surfactant, when used, makes up from 0.01 to 6%, and preferably, from 0.1 to 3% by weight of the total weight of cationic polymer and cationic surfactant used in the wash composition.

As to the anionic surfactant suitable for use in the present invention, the same comprises from 25 to 100%, and preferably, from 30 to 100%, and most preferably, from 50 to 100% by weight of a taurate surfactant based on total weight of the anionic surfactant in the composition. In still another embodiment of the present invention, the anionic surfactant is 85 to 100% by weight taurate comprising surfactant. In yet another embodiment, taurate surfactant makes up from 92 to 100%, and preferably, 100% by weight of the anionic surfactant in the composition.

The taurate surfactants suitable for use can include acylamides of taurine like N-methyltaurine, and salts thereof. Additional taurates suitable for use include those commonly known as sodium methyl lauroyl taurate, potassium methyl lauroyl taurate, sodium methyl myristoyl taurate, potassium methyl myristoyl taurate, ammonium methyl myristoyl taurate, sodium methyl cocoyl taurate, potassium methyl cocoyl taurate, ammonium methyl cocoyl taurate, sodium methyl oleoyl taurate, potassium methyl oleoyl taurate, ammonium methyl oleoyl taurate, sodium lauroyl taurate, potassium lauroyl taurate, ammonium myristoyl taurate, sodium cocoyl taurate, potassium oleoyl taurate, a mixture thereof or the like.

In a preferred embodiment, the anionic surfactant used in the invention is sodium methyl lauroyl taurate, sodium methyl cocoyl taurate or a mixture thereof. Typically, anionic surfactant makes up from 1 to 7%, and preferably, 1.5 to 6% and most preferably, from 2 to 5% by weight of the wash composition, including all ranges subsumed therein.

Optional anionic surfactants suitable for use (i.e., when anionic surfactant is not 100% taurate) include aliphatic sulfonates, such as a primary alkane (e.g., C₈-C₂) sulfonate, primary alkane (e.g., C₈-C₂) disulfonate, C₈-C₂₂ alkene sulfonate, C₈-C₂₂ hydroxyalkane sulfonate or alkyl glyceryl ether sulfonate (AGS); or aromatic sulfonates such as alkyl benzene sulfonate. The optional anionic may also be an alkyl sulfate (e.g., C₁₂-C₁₈ alkyl sulfate) or alkyl ether sulfate (including alkyl glyceryl ether sulfates). Anionic surfactant used may also include alkyl sulfosuccinates (including mono- and dialkyl, e.g., C₆-C₂₂ sulfosuccinates); alkyl and acyl sarcosinates, sulfoacetates, C₈-C₂₂ alkyl phosphates and phosphonates, alkyl phosphate esters and alkoxyl alkyl phosphate esters, acyl lactates, C₈-C₂₂ monoalkyl succinates and maleates, sulphoacetates, alkyl glucosides and acyl isethionates, mixtures thereof or the like.

The isethionates that may be used along with the taurates include C₈-C₁₈ acyl isethionates (including those which have a substituted head group). These esters are prepared by a reaction between alkali metal isethionate with mixed aliphatic fatty acids having from 6 to 18 carbon atoms and an iodine value of less than 20. Often at least 75% of the mixed fatty acids have from 12 to 18 carbon atoms and up to 25% have from 6 to 10 carbon atoms.

The acyl isethionate used may be an alkoxylated isethionate such as is described in Ilardi et al., U.S. Pat. No. 5,393,466, entitled “Fatty Acid Esters of Polyalkoxylated isethonic acid; issued Feb. 28, 1995; hereby incorporated by reference. Branched isethionates are also suitable for use

In an embodiment of the invention, an anionic surfactant used in addition to a taurate is sodium lauroyl glycinate, sodium cocoyl glycinate, sodium lauroyl glutamate, sodium cocoyl glutamate, sodium lauroyl isethionate, sodium cocoyl isethionate or a mixture thereof. Such anionic surfactants are commercially available from suppliers like Galaxy Surfactants, Clariant, Sino Lion and Innospec. Sodium cocoyl isethionate, sodium methyl lauroyl isethionate or mixtures thereof are often preferred anionics suitable for use with taurate surfactants present in the wash composition of the invention.

The amphoteric and/or zwitterionic surfactant, and nonionic surfactant or cationic polymer or mixture thereof (“component i and ii”, respectively) to anionic surfactant (“component iii”) are at a weight ratio from 1:4 to 2:1, and preferably, 1:3 to 1.5:1, and most preferably, from 1:2.5 to 1.35:1, including all weight ratios subsumed therein. The amphoteric and/or zwitterionic surfactant (“component i”) to nonionic surfactant, or cationic polymer or mixture thereof (“component ii”) are at a weight ratio from 1:12 to 12:1, and preferably, from 1:8 to 8:1, and most preferably, from 1:6 to 6:1, including all weight ratios subsumed therein.

As to the polyol suitable for use in the present invention, these include, for example, sorbitol, glycerol, mannitol, xylitol, maltitol or a mixture thereof. In an embodiment of the invention, the polyol used is at least 50% by weight glycerol, based on total weight of the polyol used in the wash composition. In another embodiment of the invention, the polyol used is all glycerol, 100% by weight, based on total weight of polyol in the wash composition. Polyol will typically make up from greater than 5 to 55% by weight of the wash composition, and preferably, from 10 to 30% by weight of the wash composition, and most preferably, from 10 to 25% by weight of the wash composition, including all ranges subsumed therein.

Water will typically make up from 50 to 96%, and preferably, from 60 to 94%, and preferably, from 65 to 92% by weight of the composition, including all ranges subsumed therein.

Adjusters suitable to modify/buffer the pH may be used. Such pH adjusters include triethylamine, NaOH, KOH, H₂SO₄, HCl, Ce He O₇ (i.e., citric acid) or mixtures thereof. The pH adjusters are added at amounts to yield the desired final pH. The pH values may be assessed with commercial instrumentation such as a pH meter made commercially available from Thermo Scientific®.

Optional skin benefit agents suitable for use in the wash composition of this invention are limited only to the extent that they are capable of being topically applied, and suitable to dissolve in the wash composition at the desired pH.

Illustrative examples of the benefit agents suitable to include in the water portion of the wash composition are acids, like amino acids, such as arginine, valine or histidine. Additional water-soluble benefit agents suitable for use include vitamin B₂. niacinamide (vitamin B₃), vitamin Be, vitamin C, mixtures thereof or the like. Water soluble derivatives of such vitamins may also be employed. For instance, vitamin C derivatives such as ascorbyl tetraisopalmitate, magnesium ascorbyl phosphate and ascorbyl glycoside may be used alone or in combination with each other. Other water-soluble benefit agents suitable for use include 4-ethyl resorcinol, extracts like sage, aloe vera, green tea, grapeseed, thyme, chamomile, yarrow, cucumber, liquorice, rosemary extract, 4-ethyl resorcinol or mixtures thereof. Water soluble sunscreens like ensulizole may also be used. Total amount of optional water-soluble benefit agents (including mixtures) when present in the invention may range from 0.0 to 10%, preferably from 0.001 to 8%, and most preferably, from 0.01 to 6% by weight of the wash composition, based on total weight of the wash composition and including all ranges subsumed therein.

It is also within the scope of the present invention to optionally include oil (i.e., non-water) soluble benefit agents. The wash composition is substantially free of oil (i.e, from 0.0 to 5%, and preferably, from 0.01 to 3.5%, and most preferably, from 0.1 to 2.5% by weight oil) and preferably where oil is not meant to include any oil added from a fragrance. Thus, oil soluble actives or benefit agents are typically solubilized in the surfactants used in the wash composition. The only limitation with respect to such oil soluble benefit agents are that the same are suitable to provide a benefit when topically applied.

Illustrative examples of the types of oil soluble benefit agents that may optionally be used in the compositions of this invention include components like stearic acid, vitamins like Vitamin A, D, E and K (and their oil soluble derivatives), sunscreens like ethylhexylmethoxycinnamate, bis-ethyl hexyloxyphenol methoxyphenol triazine, 2-ethylhexyl-2-cyano-3,3-diphenyl-2-propanoic acid, drometrizole trisiloxane, 3,3,5-trimethyl cyclohexyl 2-hydroxybenzoate, 2-ethylhexyl-2-hydroxybenzoate or mixtures thereof.

Other optional oil soluble benefit agents suitable for use include resorcinols like 4-hexyl resorcinol, 4-phenylethyl resorcinol, 4-cyclopentyl resorcinol, 4-cyclohexyl resorcinol 4-isopropyl resorcinol or a mixture thereof. Also, 5-substituted resorcinols like 4-cyclohexyl-5-methylbenzene-1,3-diol, 4-isopropyl-5-methylbenzene-1,3-diol, mixtures thereof or the like may be used. The 5-substituted resorcinols, and their synthesis are described in commonly assigned U.S. Published Patent Application No. 2016/0000669A1.

Even other oil soluble actives suitable for use include omega-3 fatty acids, omega-6 fatty acids, climbazole, farnesol, ursolic acid, myristic acid, geranyl geraniol, oleyl betaine, cocoyl hydroxyethyl imidazoline, hexanoyl sphingosine, 12-hydroxystearic acid, petroselinic acid, conjugated linoleic acid, terpineol, thymol mixtures thereof or the like.

In an embodiment of the invention, the optional oil soluble benefit agent used is a retinoic acid precursor. In one embodiment of the invention, the retinoic acid precursor is retinol, retinal, retinyl propionate, retinyl palmitate, retinyl acetate or a mixture thereof. Retinyl propionate, retinyl palmitate and mixtures thereof are typically preferred.

When optional oil soluble benefit agent is used in the wash composition of the invention, such benefit agent typically makes up from 0.0 to 0.75%, and preferably, from 0.001 to 5%, and most preferably, from 0.05 to 0.35% by weight of the wash composition. In yet another embodiment, oil makes up from 0.1 to 0.6% by weight of the total weight of the wash composition, including all ranges subsumed therein and excluding any oil provided from a fragrance.

Conventional preservatives can desirably be incorporated into the wash composition to protect against the growth of potentially harmful microorganisms. Cosmetic chemists are familiar with appropriate preservatives and routinely choose them to satisfy the preservative challenge test and to provide product stability. Suitable traditional preservatives for use include hydantoin derivatives and propionate salts. Particularly preferred preservatives are sodium benzoate, iodopropynyl butyl carbamate, phenoxyethanol, hydroxyacetophenone, ethylhexylglycerine, hexylene glycol, methyl paraben, propyl paraben, imidazolidinyl urea, sodium dehydroacetate, dimethyl-dimethyl (DMDM) hydantoin and benzyl alcohol and mixtures thereof. Other preservatives suitable for use include sodium dehydroacetate, chlorophenesin and decylene glycol. The preservatives should be selected having regard for the use of the composition and possible incompatibilities between the preservatives and other ingredients in the emulsion. Preservatives are preferably employed in amounts ranging from 0.01% to 2.0% by weight of the total weight of the wash composition, including all ranges subsumed therein. Also preferred is a preservative system with hydroxyacetophenone alone or in a mixture with other preservatives as well as preservative systems that use from 0.2 to 1.0% by weight 1,2-octanediol.

Fragrances, fixatives, chelators (like EDTA), opacifiers (like titanium dioxide), and exfoliants may optionally be included in the wash composition of the present invention. Each of these substances may range from about 0.03 to about 4%, preferably between 0.1 and 2% by weight of the total weight of the wash composition, including all ranges subsumed therein. To the extent the exfoliants are used, those selected should be of small enough particle size so that they do not impede the performance of any packaging used to dispense the compositions of this invention.

Conventional emulsifiers having an HLB of greater than 8 may optionally be used. Illustrative examples include Tween, 40, 60, 80, polysorbate 20 and mixtures thereof. Typically, emulsifiers for water continuous systems, when used, make up from 0.03 to 1.5% by weight of the wash composition.

As to the wash composition of the present invention, the same typically has from 1 to 20%, and preferably, from 2 to 18%, and most preferably, from 3 to 12% by weight total surfactant, based on total weight of the wash composition and including all ranges subsumed therein. In an embodiment of the invention, the wash composition comprises from 4.5 to 11% by weight total surfactant based on total weight of the wash composition and including all ranges subsumed therein.

Yet another optional additive suitable for use includes hemp oil with 2.5 to 25% by weight cannabigerol and/or cannabidiol at from 0.5 to 10 percent by weight. When used, such oil makes up from 0.0001 to 1.5% by weight of the wash composition, and preferably, from 0.01 to 1% by weight of the wash composition, including all ranges subsumed therein.

Even additional optional additives suitable for use in the wash composition of the invention includes eucalyptus oil, lavender oil, N,N-diethyl-meta-toluamide (DEET), octopirox (piroctone), zinc pyrithione, benzethonium chloride, benzalkonium chloride, cetrimonium chloride, benzalkonium chloride, chloroxylenol, triclosan, cetylpyridinium chloride, silver oxide, silver nitrate, silver sulfate, silver phosphate, silver carbonate, silver acetate, silver benzoate or a mixture thereof. If used, such additives typically and collectively make up from 0.0001 to 5%, and from 0.001 to 4%, and most preferably, from 0.01 to 2.5% by weight of the wash composition, including all ranges subsumed therein.

Typically, the viscosity of the wash composition will be under 20,000 cps. Often the viscosity of the wash composition will be from 10 to 15,000, and preferably, from 20 to 10,000, and most preferably, from 20 to 8,000 cps, including all ranges subsumed therein. In an embodiment of the invention, the viscosity of the wash composition is from 25 to 6,000 cps, and in still another embodiment of the invention, the viscosity of the wash composition is from 50 to 5,000 cps.

When making wash composition of the present invention, the desired ingredients may be mixed with conventional apparatus under moderate shear and atmospheric conditions, with temperature being from 30 to 85° C. whereby shear continues until a homogeneous product is recovered.

The packaging for the wash composition typically is not limited as long as composition can be dispensed. In an embodiment on the invention, the wash composition is sold in a pouch, bottle (including spray bottle), jar, tube, towelette (such as a wipe and preferably a biodegradable wipe) or canister. The packaging preferably allows for infinite numbers of refilling to invariably reduce plastic waste in the environment. In an often desired embodiment, the packaging is biodegradable and/or made of at least 50%, and preferably, at least 75% by weight post-consumer resin.

The Examples are provided to facilitate an understanding of the invention. They are not intended to limit the scope of the claims.

### Example I

Compositions were made by mixing the ingredients listed in the tables with moderate shear, under conditions of atmospheric pressure and at a temperature of about 50° C. Mixing continued until a homogeneous composition was recovered. All compositions were assessed for eye irritation (i.e., redness) as described below. Bulbar, palpebral and lacrimation assessments were made by a medical doctor, a specialist in ophthalmology. The assessments from the panellist were based on panellist self-perception and visual examination after contact with composition at the noted times. Redness refers to redness of the bulbar conjunctiva and palpebral conjunctiva.

Instillation of product (5% solution) in the ocular region of panellists and assessment protocol were carried out as described in Khanna et al., Human Ocular Response to Instillation of Surfactant Solutions and Water Across 10,000 Subjects, Altex Proceedings, 1/12, Proceedings of WC8 (2012)

Composition A

The formulation used and identified as Composition A was commercially available Johnson's® Baby Shampoo that contains Water, Cocamidopropyl Betaine, Coco-Glucoside, Sodium Methyl 2-Sulfolaurate/Disodium 2-Sulfolaurate, Citric Acid, Cetyl Betaine, Glycerin, Polyquaternium-7, Sodium Benzoate, and Parfum. Such product was used as it is advertised as a no irritation and no tear baby wash composition.

Composition B

Wash Formulation Control*

| INCI Name | % Active |
|---|---|
| Cocamidopropyl Betaine | 1.25 |
| Sodium methyl cocoyl taurate | 3.75 |
| Glycerin | 20 |
| Xanthan gum | 0.8 |
| Cationic hydroxyethylcellulose** | 0.06 |
| Tetrasodium EDTA | 0.05 |
| Preservative | 0.6 |
| pH Adjuster | 0.08 |
| Fragrance | 0.8 |
| Water | Balance |

*Conventional composition made inconsistent with the present invention.
**UCARE Polymer JR-30M

Composition C

Inventive Wash Formulation, Nonionic Surfactant Included*

| INCI Name | % Active |
|---|---|
| Cocamidopropyl Betaine | 1.25 |
| Sodium methyl cocoyl taurate | 2.5 |
| Glycerin | 20 |
| Xanthan gum | 0.8 |
| Cationic hydroxyethylcellulose** | 0.4 |
| Capryloyl/Capryl Methyl Glucamide*** | 1.6 |
| Preservative | 0.6 |
| pH Adjuster | 0.08 |
| Fragrance | 0.8 |
| Water | Balance |

*Cationic polymer present at 7.5% of the total weight of surfactant, Nonionic present.
**UCARE Polymer JR-400;
***GlucoTain ®

Composition D

Inventive Wash Formulation, Cationic Polymer*

| Ingredient | % Active |
|---|---|
| Cocamidopropyl Betaine | 1.25 |
| Sodium methyl cocoyl taurate | 3.75 |
| Glycerin | 20 |
| Xanthan gum | 0.8 |
| Guar Hydroxypropyl trimonium chloride** | 0.4 |
| Tetrasodium EDTA | 0.05 |
| Preservative | 0.6 |
| pH Adjuster | 0.03 |
| Fragrance | 0.8 |
| Water | Balance |

*Cationic polymer present at 8% of the total weight of surfactant, nonionic not present.
**UCARE Polymer JR-30M

Passing Criteria

Passing is defined to mean no score of 3 after assessment at immediate use/contact or at the 15-minute interval, and all endpoints scored as 0 (i.e., no failure after panelist use) at the 60-minute time-point, and 100% of lacrimation scores at the 15-minute time point are 0 (none) and/or 1 (mild) after all assessments of the panelists.
0—Pass—no visual irritation, discomfort, redness and/or watery eyes, lacrimation or epiphora.
1—Pass—very mild to negligible irritation, discomfort, visual redness and/or watery eyes, lacrimation or epiphora.
2—Pass—irritation, visual redness (Bulbar and Palpebral).
3—Fail, severe visual irritation, and/or severe discomfort and/or severe level of watery eyes, lacrimation and epiphora.

### Example II

The numbers to the right of the treatment (i.e., Product. Water for control in the eye not subjected to product) depict the number of panelists of a total of 25 that gave the score for the control product (Composition A). The balance of panelists means that number of panelists would have given or been assessed to have a high pass score of zero (0).

Composition A, Johnson's Baby Shampoo ®

| | | Treatment | | | |
|---|---|---|---|---|---|
| Eye Portion, time of assessment | | & Score | 1 | 2 | 3 |
| Bulbar | 30 Seconds | Product | 9 | 0 | 0 |
| | Post Instillation | Water | 0 | 0 | 0 |
| | 15 Minutes | Product | 16 | 4 | 0 |
| | Post Instillation | Water | 0 | 0 | 0 |
| | 60 Minutes | Product | 2 | 0 | 0 |
| | Post Instillation | Water | 0 | 0 | 0 |
| Palpebral | 30 Seconds | Product | 0 | 0 | 0 |
| | Post Instillation | Water | 0 | 0 | 0 |
| | 15 Minutes | Product | 8 | 0 | 0 |
| | Post Instillation | Water | 0 | 0 | 0 |
| | 60 Minutes | Product | 0 | 0 | 0 |
| | Post Instillation | Water | 0 | 0 | 0 |
| Lacrimation | 30 Seconds | Product | 4 | 0 | 0 |
| | Post Instillation | Water | 4 | 0 | 0 |
| | 15 Minutes | Product | 1 | 0 | 0 |
| | Post Instillaton | Water | 0 | 0 | 0 |
| | 60 Minutes | Product | 0 | 0 | 0 |
| | Post Instillation | Water | 0 | 0 | 0 |
| Subjective | 30 Seconds | Product | 3 | 0 | 0 |
| | Post Instillation | Water | 0 | 0 | 0 |
| | 15 Minutes | Product | 4 | 0 | 0 |
| | Post Instillation | Water | 0 | 0 | 0 |
| | 60 Minutes | Product | 0 | 0 | 0 |
| | Post Instillation | Water | 0 | 0 | 0 |

### Example III

Assessment of Composition B was terminated. The composition was not made according to the present invention, and after commencing assessment with four (4) panelists, three panelists immediately failed the assessment study.

For Compositions C and D the numbers to the right of the treatment (i.e., Product. Water) depict the number of panelists of a total of 15 that gave the score for these products. The balance of panelists means that number of panelists would have given or been assessed to have a high pass score of zero (0).

### Example IV

Composition C, Nonionic Present

| Eye Portion, time of assessment | | Treatment | 1 | 2 | 3 |
|---|---|---|---|---|---|
| Bulbar | 30 Seconds Post | Product | 2 | 0 | 0 |
| | Instillation | Water | 2 | 0 | 0 |
| | 15 Minutes Post | Product | 0 | 0 | 0 |
| | Instillation | Water | 0 | 0 | 0 |
| | 60 Minutes Post | Product | 0 | 0 | 0 |
| | Instillation | Water | 0 | 0 | 0 |
| Palpebral | 30 Seconds Post | Product | 1 | 0 | 0 |
| | Instillation | Water | 1 | 0 | 0 |
| | 15 Minutes Post | Product | 0 | 0 | 0 |
| | Instillation | Water | 0 | 0 | 0 |
| | 60 Minutes Post | Product | 0 | 0 | 0 |
| | Instillation | Water | 0 | 0 | 0 |
| Lacrimation | 30 Seconds Post | Product | 1 | 0 | 0 |
| | Instillation | Water | 4 | 0 | 0 |
| | 15 Minutes Post | Product | 2 | 0 | 0 |
| | Instillation | Water | 3 | 0 | 0 |
| | 60 Minutes Post | Product | 0 | 0 | 0 |
| | Instillation | Water | 1 | 0 | 0 |
| Subjective | 30 Seconds Post | Product | 3 | 0 | 0 |
| | Instillation | Water | 1 | 0 | 0 |
| | 15 Minutes Post | Product | 3 | 0 | 0 |
| | Instillation | Water | 0 | 0 | 0 |
| | 60 Minutes Post | Product | 0 | 0 | 0 |
| | Instillation | Water | 0 | 0 | 0 |

### Example V

Composition D, No Nonionic Present

| Eye Portion, time of assessment | | Treatment | 1 | 2 | 3 |
|---|---|---|---|---|---|
| Bulbar | 30 Seconds Post | Product | 1 | 0 | 0 |
| | Instillation | Water | 0 | 0 | 0 |
| | 15 Minutes Post | Product | 2 | 0 | 0 |
| | Instillation | Water | 0 | 0 | 0 |
| | 60 Minutes Post | Product | 0 | 0 | 0 |
| | Instillation | Water | 0 | 0 | 0 |
| Palpebral | 30 Seconds Post | Product | 0 | 0 | 0 |
| | Instillation | Water | 0 | 0 | 0 |
| | 15 Minutes Post | Product | 0 | 0 | 0 |
| | Instillation | Water | 0 | 0 | 0 |
| | 60 Minutes Post | Product | 0 | 0 | 0 |
| | Instillation | Water | 0 | 0 | 0 |
| Lacrimation | 30 Seconds Post | Product | 3 | 0 | 0 |
| | Instillation | Water | 2 | 0 | 0 |
| | 15 Minutes Post | Product | 1 | 0 | 0 |
| | Instillation | Water | 1 | 0 | 0 |
| | 60 Minutes Post | Product | 0 | 0 | 0 |
| | Instillation | Water | 0 | 0 | 0 |
| Subjective | 30 Seconds Post | Product | 4 | 1 | 0 |
| | Instillation | Water | 0 | 0 | 0 |
| | 15 Minutes Post | Product | 3 | 0 | 0 |
| | Instillation | Water | 0 | 0 | 0 |
| | 60 Minutes Post | Product | 0 | 0 | 0 |
| | Instillation | Water | 0 | 0 | 0 |

As observed with the inventive composition having surfactant and cationic polymer consistent with the present invention, surprisingly a non-irritating composition was derived. Moreover, trained panelists that washed with the compositions of the present invention concluded that the compositions had excellent lathering characteristics.

We claim:

1. A composition comprising:
a) a surfactant component comprising:
i) amphoteric and/or zwitteronic surfactant;
ii) nonionic surfactant, cationic polymer or a mixture thereof; and
iii) anionic surfactant the anionic surfactant comprising from 25 to 100% by weight taurate based on total weight of anionic surfactant;
iv) from greater than 5 to 55% by weight polyol; and
v) water
wherein the amphoteric and/or zwitterionic surfactant, and nonionic surfactant or cationic polymer or mixture thereof (“component i and ii”, respectively) to anionic surfactant (“component iii”) are at a weight ratio from 1:4 to 2:1 and the amphoteric and/or zwitterionic surfactant (“component i”) to nonionic surfactant, or cationic polymer or mixture thereof (“component ii”) are at a weight ratio from 1:12 to 12:1, with the proviso that when cationic polymer is used alone or with nonionic surfactant, the total weight of cationic polymer used to total weight of surfactant in the wash composition is from 1.3% to no more than 15% by weight of total weight of surfactant and the wash composition comprises 20% or less by weight total of surfactant and cationic polymer and further wherein the amphoteric surfactant comprises a betaine, the cationic polymer comprises hydroxypropyl trimonium chloride and/or hydroxyethylcellulose and the nonionic surfactant comprises a glucamide.

2. The composition according to claim 1 wherein the amphoteric surfactant is present and is a sodium acyl amphoacetate, a sodium acyl amphopropionate or a mixture thereof.

3. The composition according to claim 1 wherein the amphoteric surfactant is present and is sodium lauroamphoacetate, sodium cocoamphoacetate, potassium lauroamphoacetate, potassium cocoamphoacetate or mixtures thereof.

4. The composition according to claim 1 wherein the amphoteric surfactant is present and is sodium lauroamphoacetate.

5. The composition according to claim 1 wherein amphoteric surfactant makes up from 0.5 to 7% by weight of the composition.

6. The composition according to claim 1 wherein zwitterionic surfactant is present and is lauryl betaine, betaine citrate, cocodimethyl carboxymethyl betaine, cocamidopropyl betaine, cocoalkyldimethyl betaine, and laurylamidopropyl betaine or a mixture thereof.

7. The composition according to claim 1 wherein zwitterionic surfactant is present and is cocamidopropyl betaine, cocamidopropyl sultaine, cocamidopropyl hydroxysultaine, sodium hydroxymethylglycinate, (carboxymethyl) dimethyl-3-[(1-oxododecyl) amino] propylammonium hydroxide, (carboxymethyl) dimethyloleylammonium hydroxide, cocamidopropyl betaine, (carboxymethyl) dimethyloleylammonium hydroxide, cocoamidopropyl betaine, (carboxylatomethyl) dimethyl(octadecyl)ammonium, cocamidopropyl hydroxysultaine, cocoalkyldimethyl betaine, cocodimethyl carboxymethyl betaine, cocamidopropyl betaine, laurylamidopropyl betaine or a mixture thereof.

8. The composition according to claim 1 wherein zwitterionic surfactant is present and is cocamidopropyl betaine, cocamidopropyl hydroxysultaine or a mixture thereof.

9. The composition according to claim 1 wherein zwitterionic surfactant makes up from 0.5 to 7% by weight of the composition.

10. The composition according to claim 1 wherein the zwitterionic surfactant is present and is lauryl betaine, betaine citrate, cocodimethyl carboxymethyl betaine, laurylamidopropyl betaine, cocamidopropyl betaine, cocamidopropyl sultaine, cocamidopropyl hydroxysultaine, lauryl betaine, betaine citrate, sodium hydroxymethylglycinate, (carboxymethyl) dimethyl-3-[(1-oxododecyl) amino] propylammonium hydroxide, cocoalkyldimethyl betaine, (carboxymethyl) dimethyloleylammonium hydroxide, (carboxymethyl) dimethyloleylammonium hydroxide, cocoamidopropyl betaine, (carboxylatomethyl) dimethyl(octadecyl)ammonium, cocamidopropyl hydroxysultaine, or a mixture thereof.

11. The composition according to claim 1 wherein zwitterionic surfactant makes up from 1 to 6%, by weight of the composition.

12. The composition according to claim 1 wherein the composition comprises sodium lauroamphoacetate, cocamidopropyl betaine, cocamidopropyl hydroxysultaine or a mixture thereof, and a glucamide, polyglycerol ester or both.

13. The composition according to claim 1 wherein the polyol is glycerol and present at 10 to 30% by weight of the composition.

14. The composition according to claim 1 wherein the either cationic polymer or nonionic surfactant is present, and the composition is a baby wash, 2-in-1 composition or 3-in-1 composition.

15. The composition according to claim 1 wherein the nonionic surfactant is lauroyl methyl glucamide, myristoyl methyl glucamide, cocoyl methyl glucamide, capryloyl/caproyl methyl glucamide, sunfloweroyl methyl glucamide, polyglyceryl-8 caprylate, polyglycerol-8 caprate, polyglyceryl-8 laurate, polyglyceryl-8 myristate, polyglyceryl-8 palmitate, polyglycerol-8 stearate, polyglycerol-8 oleate, polyglyceryl-8 cocoate, polyglyceryl-9 caprylate, polyglycerol-9 caprate, polyglyceryl-9 laurate, polyglyceryl-9 myristate, polyglyceryl-9 palmitate, polyglycerol-9 stearate, polyglycerol-9 oleate, polyglyceryl-9 cocoate, polyglycerol-10 caprylate, polyglycerol-10 caprate, polyglycerol-10 laurate, polyglycerol-10 myristate, polyglycerol-10 palmitate, polyglycerol-10 stearate, polyglycerol-10 oleate, polyglyceryl-10 cocoate, polyglycerol-11 caprylate, polyglycerol-11 caprate, polyglycerol-11 laurate, polyglycerol-11 myristate, polyglycerol-11 palmitate, polyglycerol-11 stearate, polyglycerol-11 oleate, polyglyceryl-11 cocoate, polyglycerol-12 caprylate, polyglycerol-12 caprate, polyglycerol-12 laurate, polyglycerol-12 myristate, polyglycerol-12 palmitate, polyglycerol-12 stearate, polyglycerol-12 oleate, polyglyceryl-12 cocoate or a mixture thereof.

16. The composition according to claim 1, wherein the amphoteric surfactant is a betaine and is cocoylamidopropyl betaine, the nonionic surfactant is capryloyl/capryl methyl glucamide and the cationic polymer is hydroxypropyl trimonium chloride and/or hydroxyethylcellulose and the composition does comprise xanthan gum.
